(19) United States
(12) Patent Application Publication
(10) Pub. No.: US 2016/0340730 A1
(43) Pub. Date: Nov. 24, 2016

(54) METHODS FOR EVALUATING NEUROLOGICAL DISEASE

(71) Applicant: DIOGENIX, INC., Gaithersburg, MD (US)

(72) Inventors: Lei BAO, Gaithersburg, MD (US); Douglas W. BIGWOOD, Gaithersburg, MD (US)

(21) Appl. No.: 15/106,784

(22) PCT Filed: Dec. 19, 2014
(86) PCT No.: PCT/US14/71517

§ 371 Date: Jun. 20, 2016

Related U.S. Application Data
(60) Provisional application No. 61/918,919, filed on Dec. 20, 2013.

(51) Int. Cl. C12Q 1/68; G06F 19/22

(52) U.S. Cl. CPC C12Q 1/6883; G06F 19/22; C12Q 2600/156; C12Q 2600/112

(57) ABSTRACT

The present invention provides methods and systems for evaluating neurological disease in a patient. The neurological disease may be a demyelinating disease such as multiple sclerosis. The invention provides convenient and non-invasive genetic-based tests for evaluating a patient for demyelinating disease, including for diagnosing demyelinating disease, for excluding demyelinating disease as a diagnosis, for determining the presence of disease activity associated with demyelinating disease, and for monitoring the course of disease or efficacy of treatment for demyelinating disease.

### CROSS-REFERENCE TO RELATED APPLICATIONS

This application claims the benefit of U.S. Provisional Application Ser. No. 61/918,919, filed Dec. 20, 2013, which is herein incorporated by reference in its entirety.

### FIELD OF THE PRESENT INVENTION

The present invention generally relates to the field of neurological disease and the ability to discern mutation profiles of patient genetic sequences to ascertain the presence or absence of neurological disease, including demyelinating diseases.

### BACKGROUND OF THE PRESENT INVENTION

Disorders under the umbrella of neuroimmunology are not just the prototypic immune-mediated central (CNS) and peripheral nervous system (PNS) disorders, such as multiple sclerosis and myasthenia gravis (Coyle, Front Neurol., Vol. 2: 37, 2011). Neuroimmunological disorders represent a spectrum of diseases that can affect both the CNS and the PNS. Immune-mediated neurological disorders encompass a spectrum of diseases, disorders, and syndromes. Examples include the spectrum of disorders resulting from immune reactivity to synapse components, and paraneoplastic disorders. Virtually all major neurological conditions are now recognized to have immune/inflammatory components. Immunological therapeutic strategies are being tested in many disorders, including Alzheimer's disease, cerebrovascular disease, epilepsy, and Parkinson's disease.

The immune-mediated neurological diseases, as a group, present many formidable challenges. Individually, the incidence of these diseases is rare, and as a result, there is often little awareness or understanding of them among both the general public and many physicians. Given the lack of awareness or understanding of the diseases, they become a challenge to diagnose. The challenges in accurate diagnoses are exacerbated due to the diseases often developing in the absence of antecedent warning signs, and progressing at great speed as some patients develop severe illnesses almost instantaneously. Consequently, patients have little time to make decisions, and clinicians and researchers have little opportunity to intervene.

Immune-mediated neurological diseases can be challenging to diagnose. The diagnostic approaches between hospital to hospital and practice group to practice group are often variable. The accurate diagnoses of these diseases requires detailed clinical evaluation and appropriately targeted diagnostic testing, such as neurophysiologic testing, neuro-imaging, blood tests, and sometimes cerebrospinal fluid assessment. A single diagnostic method for determining the presence or absence of immune-mediated neurological disease is not available to patients.

One of the most studied immune-mediated neurological diseases is multiple sclerosis (MS). MS is a disease that affects the central nervous system, and can range from relatively benign to somewhat disabling to devastating. In MS, the myelin surrounding nerve cells is damaged or destroyed, impacting the ability of the nerves to conduct electrical impulses to and from the brain, and leaving scar tissue called sclerosis. These damaged areas are also known as “plaques” or “lesions.”

The first symptoms of MS typically appear between the ages of 20 and 40, and include blurred or double vision, red-green color distortion, or even blindness in one eye. Most MS patients experience muscle weakness in their extremities and difficulty with coordination and balance. In severe cases, MS can produce partial or complete paralysis. Paresthesias (numbness, prickling, or “pins and needles”), speech impediments, tremors, and dizziness are frequent symptoms of MS. Approximately half of MS patients experience cognitive impairments.

Diagnosing MS is complicated, because there is no single test that can confirm the presence of MS. The process of diagnosing MS typically involves criteria from the patient's history, a clinical examination, and one or more laboratory tests, with all three often being necessary to rule out other possible causes for symptoms and/or to gather facts sufficient for a diagnosis of MS.

Magnetic resonance imaging (MRI) is a preferred test. An MRI can detect plaques or scarring in a patient's CNS tissue possibly caused by MS. However, an abnormal MRI does not necessarily indicate MS, as lesions in the brain may he associated with other disorders. Further, spots may also be found in healthy individuals, particularly in healthy older persons. These spots are called UBOs, for unidentified bright objects, and are not related to an ongoing disease process. In addition, a normal MRI does not absolutely rule out the presence of MS. About 5% of individuals who are confirmed to have MS on the basis of other criteria, have no brain lesions detectable by MRI. These individuals may have lesions in the spinal cord or may have lesions that cannot be detected by MRI.

While a diagnosis of MS might be based on an evaluation of symptoms, signs, and the results of an MRI, additional tests may also be ordered. These include tests of evoked potential, cerebrospinal fluid, and blood. For example, cerebrospinal fluid is sampled by a lumbar puncture, and is tested for levels of immune system proteins and for the presence of an antibody staining pattern called “oligoclonal bands.” Oligoclonal bands indicate an immune response within the central nervous system and are found in the spinal fluid of 90-95% of individuals with MS. However, oligoclonal bands are also associated with diseases other than MS, and therefore the presence of oligoclonal bands alone is not definitive of MS. There is likewise no definitive blood test for MS, but blood tests can exclude other possible causes for various neurologic symptoms, such as Lyme disease, collagen-vascular diseases, rare hereditary disorders, and AIDS.

Diagnosing MS generally requires: (I) objective evidence of at least two areas of myelin loss, or demyelinating lesions, “separated in time and space” (lesions occurring in different places within the brain, spinal cord, or optic nerve-at different points in time); and (2) all other diseases that can cause similar neurologic symptoms have been objectively excluded. Until (1) and (2) are satisfied, a physician does not make a definite diagnosis of MS.

Depending on the clinical problems present when an individual sees a physician, one or more of the tests described above might be performed. Sometimes tests are performed several times over a period of months to help gather the necessary information. A definite MS diagnosis must satisfy the. McDonald criteria, named for the distinguished neurologist W. Ian McDonald who sparked society-supported efforts to make the diagnostic process for MS faster and more precise.

There are a few distinct clinical courses for MS, referred to as relapsing-remitting MS, secondary-progressive MS, progressive-relapsing MS, and primary progressive MS. Relapsing-remitting MS is characterized by clearly-defined, acute attacks (relapses), usually with full or partial recovery, and no disease progression between attacks. Secondary-progressive MS is initially relapsing-remitting but then becomes continuously progressive at a variable rate, with or without occasional relapses along the way. The disease-modifying medications are thought to provide benefit for those who continue to have relapses. Primary progressive MS may be characterized by disease progression from the beginning with few or no periods of remission. Progressive-relapsing MS is characterized by disease progression from the beginning, but with clear, acute relapses along the way.

Because immune-mediated neurological diseases can present with similar symptoms, there is a need in the art for methods that can not only diagnose immune-mediated neurological disease in a patient, but distinguish one particular type of neurological disease from another. An accurate diagnosis is required as early as possible in the disease course so that an effective treatment regimen can be prescribed. In addition, treatments that may be effective for one type of neurological disease may exacerbate or accelerate progression of another type of neurological disease. Accordingly, there is a need for more precise methods that can detect neurological disease as early as possible and provide a differential diagnosis to discern one form of neurological disease from another.

### SUMMARY OF THE PRESENT INVENTION

The present invention is based, in part, on the finding that a particular genetic profile can reliably predict the presence of neurological disease in a patient. Accordingly, the present invention provides methods for detecting neurological disease, including a demyelinating disease such as multiple sclerosis, in a patient through the novel use of convenient and non-invasive genetic tests.

In one embodiment, the present invention provides a method for detecting neurological disease, including immune-mediated neurological disease and/or demyelinating disease in a patient comprising determining in VH4 gene sequences from a patient sample, a mutation profile at a plurality of VH4 codon positions, wherein the mutation profile comprises the frequency or percentage of occurrence of a plurality of the following mutations: S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, S65G; and classifying the sample for the presence or absence of neurological disease based on the mutation profile using a computer-implemented classifier algorithm. The frequency or percentage of occurrence of at least 5, at least 10, or at least 15 of the mutations may be determined. In some embodiments, the frequency or percentage of occurrence of at least 20 or least 25 of the mutations is determined. In certain embodiments, all of the mutations are determined. In some embodiments, the mutations are determined at a plurality of VH4 codon positions selected from one or more or all of codons 17, 20, 24, 26, 27, 28, 31A, 32, 35, 36, 39, 40, 41, 44, 53, 57, 58, 60, 62, 65, 73, and 81.

In some embodiments, the neurological disease is a demyelinating disease. In certain embodiments, the demyelinating disease is multiple sclerosis (MS).

The computer-implemented classifier algorithm may, in some embodiments, be a classifier algorithm trained with the frequency or percentage of mutations in MS patients as compared to healthy controls (e.g. humans not diagnosed with a neurological disease). In other embodiments, the computer-implemented classifier may be a classifier algorithm trained with the frequency or percentage of mutations in MS patients as compared to patients with other neurological diseases, such as other immune-mediated neurological diseases. In certain embodiments, the computer-implemented classifier is a k-Nearest Neighbors (KNN) algorithm. In such embodiments, k may be at least 4, at least 5, at least 6, at least 7, or at least 8. In other embodiments, the computer-implemented classifier is a top scoring pair (TSP) algorithm.

In other embodiments, the present invention provides a method for detecting multiple sclerosis disease in a patient, comprising determining sequences at codons 17 to 81 in VH4 genes from a patient sample; calculating the frequency or percentage of occurrence of at least five mutations selected from: S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, P65G; and classifying the sample for the presence or absence of multiple sclerosis by a k-Nearest Neighbors (KNN) algorithm. K may be at least 4, at least 5, at least 6, at least 7, or at least 8. In some embodiments, the k-Nearest Neighbors algorithm is trained with the frequency or percentage of the mutations in MS patients versus healthy controls and/or patients with other neurological diseases.

In certain embodiments of the methods of the invention, the classifying step determines active disease, disease progression, or disease relapse. In other embodiments, the classifying step determines disease remission or disease control. In particular embodiments, the disease is a demyelinating disease, such as MS. The classifying step may, in some embodiments, confirm or rule out a diagnosis for a particular type of neurological disease. For instance, the classifying step can confirm MS or rule out MS.

In still other embodiments, the present invention provides a method for detecting demyelinating disease in a patient, comprising detecting in VH4 sequences from a patient sample, mutations at IGVH4-34 position 16. and IGHF4-4 at position 34, and classifying the patient as having demyelinating disease if the frequency or percentage of mutation at IGVH4-34 position 16 is greater than IGH4-4 position 34. In certain embodiments, the demyelinating disease is multiple sclerosis.

The patient sample to be analyzed in the methods of the invention can be a blood sample, a cerebrospinal fluid sample (CSF), a urine sample, or fractions thereof. In one embodiment, the patient sample is CSF. In another embodiment, the patient sample is a blood sample. In some embodiments, samples may be obtained from patients undergoing treatment for MS or patients having clinically isolated syndrome.

In some embodiments of the methods of the invention, mutations and mutation profiles can be detected by nucleic acid sequencing methods, quantitative polymerase chain reaction (PCR), hybridization assay, or endonuclease assay.

In some embodiments, the methods of the invention further comprise making a majority voting prediction based on the results of one or more classification algorithms.

An object of the present invention is to provide a convenient diagnostic test for a more Objective, definitive, and rapid diagnosis of immune-mediated neurological disorders, including MS. Another object of the invention is to provide a diagnostic test for monitoring immune-mediated neurological disorder progression, adequacy of treatment, and/or response to treatment.

Other objects of the invention will be apparent from the following description of the invention.

### BRIEF DESCRIPTION OF THE DRAWINGS

FIG. 1: A plot of features “IGHV4-34@16” and IGHV4-4@34” selected with the top scoring pair (TSP) algorithm, where each plus represents a patient classified as having other neurological disease (OND) and each circle represents a patient classified as having relapsing/remitting multiple sclerosis (RMMS). The classification rule allows for the determination of likelihood of the patient to have RMMS if the fraction of mutations at “IGHV4-34@16” is greater than that at “IGHV4-4@34” (above the diagonal line), and vice versa.

### DETAILED DESCRIPTION OF THE PRESENT INVENTION

The present invention provides convenient and non-invasive genetic-based tests for diagnosing immune-mediated neurological disease (IMND) in patients. The inventors have identified a mutational profile in antibody heavy chain genes that can be used to diagnose neurological disease, including demyelinating disease, such as multiple sclerosis (MS), in patients.

The normal immune system has the ability to generate millions of antibodies with different antigen binding abilities. The diversity is brought about by the complexities of constructing immunoglobulin molecules. These molecules consist of paired polypeptide chains (heavy and light) each containing a constant and a variable region. The structures of the variable regions of the heavy and light chains are specified by immunoglobulin V genes. The heavy chain variable region is derived from three gene segments known as VH, D and JH. In humans, there are about 100 different VH segments, over 20 D segments and six JH segments. The light chain genes have only two segments, the VL and JL segments. Antibody diversity is the result of random combinations of VH/D/JH segments with VLJL components superimposed on which are several mechanisms including junctional diversity and somatic mutation.

The germline VH genes can be separated into at least six families (VH1 through VH6) based on DNA nucleotide sequence identity of the first 95 to 101 amino acids. Members of the same family typically have less than or equal to 80% sequence identity, whereas members of different families have less than 70% identity. These families range in size from one VH6 gene to an estimated greater than 45 VH3 genes. In addition, many pseudogenes exist. A physical map of the VH locus on chromosome 14q32.13.15 has nearly been completed. It has now been estimated that the human repertoire is represented by approximately 50 functional VH segments with about an equal number of pseudogenes. The VH4 family of genes contains 9 different members: 4-04, 4-28, 4-30, 4-31, 4-34, 4-39, 4-59, 4-61, and 4-B4.

The present invention is based, in part, on the identification of mutations in certain codons of the VH4 gene sequences from a patient that are associated with the presence of neurological disease in the patient. Thus, in one embodiment, the present invention provides a method for detecting neurological disease in a patient comprising determining in VH4 gene sequences from a patient sample a mutation profile at a plurality of VH4 codon positions and classifying the sample for the presence or absence of the neurological disease based on a the mutation profile using a computer-implemented classifier algorithm. In certain embodiments, the plurality of VH4 codon positions includes codons 17 to 81. For example, the plurality of VH4 codon positions may include one or more of codons 17, 20, 24, 26, 27, 28, 31A, 32, 35, 36, 39, 40, 41, 44, 53, 57, 58, 60, 62, 65, 73, and 81. In some embodiments, the mutation profile is determined from all of VH4 codon positions 17, 20 24, 26, 27, 28, 31A, 32, 35, 36, 39, 40, 41, 44, 53, 57, 58, 60, 62, 65. 73, and 81.

The mutation profile determined from VH4 gene sequences at a plurality of codons can include the frequency or percentage of occurrence of a plurality of mutations selected from S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, and S65G. In some embodiments, the S31AL mutation encompasses a mutation at codon 31A that results in the expression of a leucine as opposed to the native serine. Thus, in some embodiments, the method for detecting neurological disease in a patient comprises determining the sequence of VH4 genes in a patient sample at a plurality of codons (e.g. codons 17 to 81) to identify mutations relative to the germline VH4 sequences and calculating the frequency or percentage of occurrence of two or more mutations selected from S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, and S65G. In certain embodiments, the mutation profile comprises the frequency or percentage of occurrence of at least five or at least ten of these specific mutations. In other embodiments, the mutation profile comprises the frequency or percentage of occurrence of at least fifteen or at least twenty of these specific mutations. In still other embodiments, the mutation profile comprises the frequency or percentage of occurrence of at least twenty five of these specific mutations. In one particular embodiment, the mutation profile comprises the frequency or percentage of occurrence of all of these specific mutations.

Neurological diseases that may be detected in patient samples utilizing the methods of the invention include immune-mediated neurological diseases (IMNDs). IMNDs include those diseases in which at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis. Other neurological diseases have recently been recognized to include an inflammatory component. For instance, Alzheimer's disease, Parkinson's disease, cerebrovascular disease, epilepsy, and CNS infection among others are now believed to involve neuroinflammation (Coyle, Front Neurol., Vol. 2: 37, 2011).

Thus, patients to be evaluated by the methods of the invention may be known to have IMND, may be suspected of having IMND on the basis of one or more IMND-like symptoms or results from one or more IMND-related clinical exams, or may be beginning or undergoing treatment for IMND. In the various aspects of the invention, the invention aids in diagnosing IMND, or excluding IMND as a diagnosis, determining IMND disease activity, or monitoring the progression of IMND or a demyelinating disease consistent with IMND, or determining efficacy of an IMND treatment.

In certain embodiments, the IMND that can be detected or diagnosed with the methods of the invention is a demyelinating disease. Demyelinating diseases include, but are not limited to, multiple sclerosis, Devic's disease neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy.

In some embodiments, the method for detecting demyelinating disease in a patient comprises detecting in VH4 sequences from a patient sample, mutations at particular codon positions in certain IGVH4 genes. For instance, in one embodiment, the method for detecting demyelinating disease in a patient comprises detecting in VH4 sequences from a patient sample mutations at IGVH4-34 codon position 16, and IGHF4-4 at codon position 34, and classifying the patient has having demyelinating disease if the frequency or percentage of mutation at IGVH4-34 codon position 16 is greater than IGH4-4 codon position 34. In some embodiments, the demyelinating disease is multiple sclerosis.

In other embodiments, the method for detecting multiple sclerosis disease in a patient comprises determining sequences at a plurality of codons in VH4 genes (e.g. codons 17 to 81) from a patient sample, calculating the frequency or percentage of occurrence of at least five mutations selected from S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, S65G; and classifying the sample for the presence or absence of multiple sclerosis by a classifier algorithm, such as the K-NN algorithm or TSP algorithm. In some embodiments, the frequency or percentage of occurrence of at least five or at least ten of these specific mutations is calculated. In other embodiments, the frequency or percentage of occurrence of at least fifteen or at least twenty of these specific mutations is calculated. In still other embodiments, the frequency or percentage of occurrence of at least twenty five of these specific mutations is calculated. In one particular embodiment, the frequency or percentage of occurrence of all of these specific mutations is calculated.

In some embodiments, the methods described herein provide for the detection or diagnosis of MS in patients presenting with immune-mediated neurological symptoms. Multiple Sclerosis is one of the most common diseases of the central nervous system (brain and spinal cord). It is an inflammatory condition associated with demyelination, or loss of the myelin sheath. Myelin, a fatty material that insulates nerves, acts as insulator in allowing nerves to transmit impulses from one point to another. In MS, the loss of myelin is accompanied by a disruption in the ability of the nerves to conduct electrical impulses to and from the brain and this produces the various symptoms of MS, such as impairments in vision, muscle coordination, strength, sensation, speech and swallowing, bladder control, sexuality and cognitive function. The plaques or lesions where myelin is lost appear as hardened, scar-like areas. These scars appear at different times and in different areas of the brain and spinal cord, hence the term “multiple” sclerosis, literally meaning many scars.

In certain embodiments of the methods, the patient is suspected of having MS. For example, the patient may be suspected of having MS on the basis of neurologic and/or immunologic symptoms consistent with MS. MS symptoms include, for example, altered sensory, motor, visual or proprioceptive system with at least one of numbness or weakness in one or more limbs, often occurring on one side of the body at a time or the lower half of the body, partial or complete loss of vision, frequently in one eye at a time and often with pain during eye movement, double vision or blurring of vision, tingling or pain in numb areas of the body, electric-shock sensations that occur with certain head movements, tremor, lack of coordination or unsteady gait, fatigue, dizziness, muscle stiffness or spasticity, slurred speech, paralysis, problems with bladder, bowel or sexual function, and mental changes such as forgetfulness or difficulties with concentration, relative to medical standards.

In certain embodiments, the patient has been diagnosed as having MS. Generally, conventional diagnosis of MS relies on two criteria. First, there must have been two attacks at least one month apart. An attack, also known as an exacerbation, flare, or relapse, is a sudden appearance of or worsening of an MS symptom or symptoms which lasts at least 24 hours. Second, there must be more than one area of damage to central nervous system myelin sheath. Damage to sheath must have occurred at more than one point in time and not have been caused by any other disease that can cause demyelination or similar neurologic symptoms.

In other embodiments, the patient to be evaluated by the methods described herein may be positive for the presence of oligoclonal bands. In these and or other embodiments, the patient may have CNS lesions characteristic of MS, which are observable on a magnetic resonance image (MRI). Magnetic resonance imaging is currently the preferred method of imaging the brain to detect the presence of plaques or scarring caused by MS. In another embodiment, the patient may exhibit evoked potentials (electrical diagnostic studies that may reveal delays in central nervous system conduction times) consistent with demyelination. In some embodiments, the patient may be undergoing treatment for MS, i.e. receiving treatment, for example, with beta-interferon, glatiramer acetate, mitoxantrone, or Natalizumab.

In some embodiments of the methods of the invention, the patient has clinically isolated syndrome (CIS). A patient with CIS has experienced a first episode of neurologic symptoms that lasts at least 24 hours and is caused by inflammation and demyelination in one or more sites in the central nervous system. The episode can be monofocal, in which the patient experiences a single neurologic sign or symptom. The episode can be multifocal, in which the patient experiences more than one sign or symptoms. In certain embodiments of the methods described herein, the likelihood that a patient with CIS will develop MS is determined. In related embodiments, the methods confirm or rule out a MS diagnosis in a patient with CIS.

On the basis of the diagnosis or prediction provided by the methods described herein, a therapeutic regimen for treating an immune-mediated neurological disorder may be started, ended, or modified. In particular, patients diagnosed as having or at risk of developing MS may be started on a therapeutic regimen for treating MS. The primary aims of therapy are returning function after an attack, preventing new attacks, and preventing disability. Therapeutic regimens for MS include, but are not limited to, immunomodulating therapy (e.g. beta-interferon), glatiramer acetate (Copaxone™), immunosuppressants (e.g. mitoxantrone), and natalizumab (marketed as Tysabri™). Where MS is excluded as a diagnosis, the patient is not administered an MS treatment.

The patient sample suitable for use in the methods of the invention can be a blood sample (e.g. whole blood sample, peripheral blood mononuclear cells), a lymphatic fluid sample, a cerebrospinal fluid sample (CSF), a urine sample, or a fraction of any of the foregoing. In some embodiments, the patient sample is a blood sample. In other embodiments, the patient sample is CSF. In still other embodiments, the patient sample is a fluid or tissue containing B cells.

Methods of determining nucleotide sequences of genes and detecting mutations are known to those of skill in the art. Some exemplary methods are described herein and include direct DNA sequencing, hybridization assays, polymerase chain reaction (PCR)-based assays, and endonuclease assays.

The identity of a nucleotide (or nucleotide pair) at a particular site (e.g., site of a mutation) may be determined by amplifying a target region(s) containing the site(s) directly from one or both copies of the gene present in the individual and the sequence of the amplified region(s) determined by conventional methods.

The target region(s) may be amplified using any oligonucleotide-directed amplification method, including but not limited to various PCR methods, such as quantitative PCR and RT-PCR, ligase chain reaction (LCR), and oligonucleotide ligation assay (OLA). Oligonucleotides useful as primers or probes in such methods should specifically hybridize to a region of the nucleic acid that contains or is adjacent to the site or sites of interest. Other known nucleic acid amplification procedures may be used to amplify the target region including transcription-based amplification systems and isothermal methods.

One or more mutations in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one mutation site may be detected at once using a set of allele-specific oligonucleotides or oligonucleotide pairs.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype for one or more mutation sites in the gene of an individual may also be determined by hybridization of one or both copies of the gene, or a fragment thereof, to nucleic acid arrays and subarrays. The arrays would contain a battery of allele-specific oligonucleotides representing each of the mutation sites to be included in the genotype or haplotype.

The identity of polymorphisms or mutations may also be determined using a mismatch detection technique, including but not limited to the RNase protection method using riboprobes and proteins which recognize nucleotide mismatches, such as the E. coli mutS protein. Alternatively, variant alleles can be identified by single strand conformation polymorphism (SSCP) analysis (Orita et al., 1989; Humphries, et al., 1996) or denaturing gradient gel electrophoresis (DGGE) (Wartell et al., 1990; Sheffield et al., 1989).

A polymerase-mediated primer extension method may also be used to identify the polymorphisms or mutations. Extended primers containing a polymorphism or mutation may be detected by mass spectrometry. Another primer extension method is allele-specific PCR. In some embodiments, a particular nuclease cleavage site may be present and detection of a particular nucleotide sequence can be determined by the presence or absence of nucleic acid cleavage.

Pairs of primers designed to selectively hybridize to nucleic acids corresponding to the variable heavy chain gene locus, variants and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is PCR, which is a technique that is well known in the art.

Primer extension, which may be used as a standalone technique or in combination with other methods (such as PCR), requires a labeled primer (usually 20-50 nucleotides in length) which is complementary to a region near the 5′ end of the gene. The primer is allowed to anneal to the RNA and reverse transcriptase is used to synthesize complementary cDNA to the RNA until it reaches the 5′ end of the RNA.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88110315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention. PCI Application WO 89/06700 (incorporated herein by reference in its entirety) disclose a nucleic acid sequence amplification scheme based on the hybridization of a promoter region/primer sequence to a target single-stranded DNA (ssDNA) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Real-time polymerase chain reaction, also called quantitative real time polymerase chain reaction (qPCR) or kinetic polymerase chain reaction, is a technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample, Two common methods of quantification are the use of fluorescent dyes that intercalate with double-stranded DNA, and modified DNA oligonucleotide probes that fluoresce when hybridized with a complementary DNA.

Frequently, real-time polymerase chain action is combined with reverse transcription polymerase chain reaction to quantify low abundance messenger RNA (mRNA), enabling a researcher to quantify relative gene expression at a particular time, or in a particular cell or tissue type. Although real-time quantitative polymerase chain reaction is often marketed as RT-PCR, it should not be confused with reverse transcription polymerase chain reaction, also known as RT-PCR.

In some embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe may be conjugated to a chromophore but may be radiolabeled. In another embodiment, the probe is conjugated to a binding partner, such as an antibody or biotin, or another binding partner carrying a detectable moiety.

In one embodiment, detection is by Southern blotting and hybridization with a labeled probe. The techniques involved in Southern blotting are well known to those of skill in the art (see Sambrook et al. 2001), Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517 5,928,862, 5,928,869, 29,227, 5,932,413 and 5,935,791, each of which is incorporated herein by reference in its entirety.

Polymorphisms or mutations in target gene sequences can also be detected by endonuclease-based assays, such as restriction fragment length polymorphism (RFLP) analysis. Other methods for detecting polymorphisms or mutations include, but are not limited to, the direct or indirect sequencing of the site or region, the use of restriction enzymes where the respective alleles of the site create or destroy a restriction site, the use of allele-specific hybridization probes, the use of antibodies that are specific for the proteins encoded by the different alleles of the polymorphism or mutation, or any other biochemical interpretation.

The most commonly used method of characterizing a polymorphism or mutation is direct DNA sequencing of the genetic locus that flanks and includes the polymorphism or mutation. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes. Next generation sequencing methods, including massively parallel signature sequencing, pyrosequencing, sequencing by ligation, etc., can also be used to detect mutations in the target VH4 gene sequences.

Any suitable method known in the art, such as those described above, can be used in the practice of the methods of the invention to determine a mutation profile or detect specific mutations in VH4 gene sequences in a patient sample. In some embodiments, the mutation profile or mutations are determined by nucleic acid sequencing of VH4 genes in the sample. In other embodiments, the mutation profile or mutations are determined by a hybridization assay. In still other embodiments, the mutation profile or mutations are determined by quantitative PCR. In yet other embodiments, the mutation profile or mutations are determined by endonuclease assay.

In some embodiments of the methods of the invention, the mutation profile determined in a patient's sample is evaluated for the presence or absence of neurological disease, including a demyelinating disease, such as MS using a computer-implemented classifier algorithm. The algorithm may entail classifying a sample based on a correlation between the mutation profile determined in the sample and the mutation profile in patients diagnosed with the neurological disease (e.g., MS) and the mutation profile in healthy controls or patients with other neurological diseases. For example, samples may be classified on the basis of frequency or percentage of a plurality of specific mutations in RIND patients (e.g. MS) versus a non-IMND population (e.g., a population of healthy controls or population of patients with other neurological diseases, e.g., other than MS). Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, Penalized Logistic Regression, and Rule-based schemes. In addition, the predictions from multiple models can be combined to generate an overall prediction. For example, a “majority rules” or “majority voting” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which is hereby incorporated by reference in its entirety.

In some embodiments of the methods of the invention, the computer-implemented classifier algorithm is a K-Nearest Neighbors algorithm. KNN is a type of instance-based learning where the function is only approximated locally and all computation is deferred until classification. Input to a KNN algorithm consists of the k closest training examples in the feature space. When the KNN algorithm is used for classification, the output is a class membership. An object is classified by a majority vote of its neighbors, with the object being assigned to the class most common among its k nearest neighbors. When the KNN algorithm is used for regression, the output is the property value for the object. This value is the average of the values of its k nearest neighbors, in certain embodiments of the methods of the invention, k is at least 4 or at least 5. In other embodiments, k is at least 6 or at least 7. In a particular embodiment, k is at least 8. In some embodiments of the methods, k is 6. In other embodiments of the methods, k is 8.

For classification and regression, the contributions of the neighbors can be weighted so that the nearer neighbors contribute more to the average than the more distant neighbors. A weighting scheme may include giving each neighbor a weight of 1/d, where d is the distance to the neighbor. Neighbors are taken from a set of objects for which the class, for KNN classification, or the object property value, for KNN regression, is known. This set of objects can be considered a training set for the algorithm. However, no training step is required. The classification accuracy of KNN may be improved if the distance metric is learned with specialized algorithms such as Large Margin Nearest Neighbor or Neighborhood components analysis.

In certain embodiments of the methods of the invention, the KNN algorithm is trained with the frequency or percentage of mutations in IMND patients versus healthy controls. In other embodiments of the methods, the KNN algorithm is trained with the frequency or percentage of mutations in IMND patients versus patients with other neurological diseases. In still other embodiments, the KNN algorithm is trained with the frequency or percentage of mutations in MS patients versus healthy controls and/or patients with other immune-mediated neurological diseases. Thus, in some embodiments, the KNN classifier algorithm determines a correlation between the mutation profile determined from the VH4 genes in a patient's sample to the mutation profile from a population of MS patients versus the mutation profile from healthy controls or patients with other neurological diseases. The training sets for the algorithm may be VH4 mutation profiles in MS patients having a particular course of the disease, such as relapse-remitting MS, secondary-progressive MS, progressive-relapsing MS, and primary progressive MS. Therefore, in some embodiments, the application of the KNN algorithm may determine the course of MS disease in a patient. Additional demographic criteria, such as age, race, gender, MS treatment, and clinical manifestation and course of MS, may be used as factors in the classifier algorithm.

In particular embodiments, the KNN algorithm is applied to a data set (e.g. mutation profiles from VH4 genes) to produce a statistically derived decision indicating whether a VH4 gene mutation profile from a patient test sample is associated with active neurological disease, disease progression, disease relapse, disease remission, or disease control. In these and other embodiments, the patient may be undergoing treatment for the neurological disease, such as MS. In other embodiments, the patient has clinically isolated syndrome and application of the KNN algorithm confirms a diagnosis of MS. In still other embodiments, the patient has clinically isolated syndrome and application of the KNN algorithm rules out a diagnosis of MS.

KNN classifiers, in their most basic forms, operate under the assumption that all features are of equal value. When irrelevant and noisy features influence the neighborhood search to the same degree as highly relevant features, the accuracy of the model may deteriorate. The use of feature weighting may be used to approximate the optimal degree of influence of individual features using a training set. Relevant features may be attributed a high weight value, whereas irrelevant features are attributed a weight value close to zero. Feature weighting can be utilized to improve classification accuracy as well as to discard features with weights below a certain threshold value, thereby increasing the resource efficiency of the classifier.

In some embodiments, the features for the KNN classifier algorithms are selected using the Linear models for Microarray Data (limma). Limma is a package for differential expression analysis of data arising from microarray experiments. The package was designed to analyze complex experiments involving comparisons between many RNA targets simultaneously while remaining reasonably easy to use for simple experiments. The data may be log-ratios, or sometimes log-intensities. Empirical Bayes and other shrinkage methods are used to borrow information across genes making the analyses stable even for experiments with small number of arrays (Smyth, 2004; Smyth et al. 2005). Limma provides input and normalization functions that support features especially useful for the linear modeling approach (Smyth. 2005. Stats for Biol. Health. P. 397-420).

In other embodiments, the features for the KNN classifier algorithms are selected using a Random Forests (RF) model. RF is an ensemble learning method for classification and regression that operates by constructing a multitude of decision trees at training time and outputting the class that is the mode of the classes output by individual trees. RF may be used to rank the importance of variables in a regression or classification problem. Features which produce large values are ranked as more important than features which produce small variables.

In certain embodiments of the methods of the invention, the computer-implemented classifier algorithm is a top scoring pair (TSP) algorithm. The TSP algorithm is a simple yet powerful parameter-free classifier that is based on relative expression ordering of gene pairs. The TSP algorithm can also be utilized for feature selection.

The methods of the invention may further comprise generating a majority voting prediction based on the outputs of multiple classifier algorithms. For instance, a majority voting prediction may be made based on the results of one or more KNN algorithms having different k values. In one embodiment, the majority voting prediction is made based on the results of a KNN algorithm with a k value of 6 and a KNN algorithm with a k value of 8. In other embodiments, a majority voting prediction may be made based on the results of a KNN algorithm and a TSP algorithm. In still other embodiments, a majority voting prediction may be based on the results of three or more algorithms, such as one or more KNN algorithms and a TSP algorithm. In certain embodiments, a majority voting prediction is made based on the results of the application of a KNN algorithm to a VH4 gene mutation profile and the results of application of a TSP algorithm to the frequency or occurrence of mutation at specific codons in particular VH4 genes, such as V4-34 and VF-4-4.

Majority vote classifiers work by producing a large number of classifications and classifying to the class receiving the largest number of votes or predictions. Majority vote classifiers include Error Correcting Output Coding (ECOC), Boosting, and Bagging. Majority vote classifiers produce essentially unbiased probability estimates for each class, and classifies to the maximum or majority.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All patent and non-patent documents referenced throughout this disclosure are incorporated by reference herein in their entirety for all purposes.

### EXAMPLES

### Example 1

Four batches of 70 patient blood samples were made available for investigation. The patient pool contained 32 patients with Relapsing/Remitting Multiple Sclerosis (RRMS), 12 patients with possible RRMS, and 26 patients with other neurological disease (OND).

Binary classifiers were built to differentiate 44 possible RRMS vs. 26 OND patients. The sample size can be significantly increased by including the 12 possible RRMS patients, at the cost of, presumably, a higher possibility of inclusion of a small number of mislabeled patients.

Two different sets of features were derived from the sequence data. The first being pSUB, the percentage of the occurrence of individual substitution event (e.g. “G44W”). The second being the pVGENE, the percentage of the occurrence of any substitution events at a specific position stratified by the IGVH4 subfamilies (e.g. “IGHV4-34@16”).

Leave one-out-cross-validation (LOOCV) was used to assess the performance. In order to be rigorous, the left-out sample is always Hind to both feature selection and classifier training process.

Two different modelling approaches were attempted: KNN and TSP (German et al., Stat. Appl. In Genetics and Molecular Biology, 2004). TSP has a built-in feature selection mechanism, while for KNN we need to look into several feature selection algorithms. The standard Student's t-test method did not work well for this dataset. Features selected by random forest (RF) did not work well either. Instead, features were selected by limma (Linnear Models for Microarray Data User's Guide).

### KNN Classifier

Two KNN classifiers using pSUB as features gave the best overall classification accuracy. KNN classifiers using pVGENE were less useful.

TABLE 1
LOOCV performance of KNN classifier

| | Overall | Sensitivity | Specificity |
|---|---|---|---|
| Classifier | Accuracy | (RRMS) | (OND) |
| K = 6, 28 features, | 84.3% (11) | 86.4% (6) | 80.8% (5) |
| (All 70 patients) | | | |
| K = 6, 28 features, | 84.5% (9) | 87.5% (4) | 80.8% (5) |
| (58 confirmed patients) | | | |
| K = 8, 28 features, | 84.3% (11) | 90.9% (4) | 73.1% (7) |
| (All 70 patients) | | | |
| K = 8, 28 features, | 84.5% (9) | 93.8% (2) | 73.1% (7) |
| (58 confirmed patients) | | | |

The number found within the parentheses in Table 1 indicates the number of misclassified samples.

The two optimal classifiers give similar overall accuracy of 84% to 85%, but differ in sensitivity and specificity. The former has quite a balanced performance between sensitivity and specificity. The latter has high sensitivity, but somewhat lower specificity. The classifier should therefore be selected based on the clinical utility, with consideration given to which side is better to err.

When focused only on RRMS patients (i.e. disregard 12 possible RRMS cases), the sensitivity and overall accuracy are marginally increased. Limiting the analysis to patients based on consensus of 3 did not improve the accuracy, but only reduced the sample size.

After LOOCV, the entire data set (all 70 patients) was used to select the 28 features.

TABLE 2
Twenty eight features selected using the entire dataset.

| Feature | Score | Feature | Score | Feature | Score |
|---|---|---|---|---|---|
| S31AL | −3.93583 | G35A | 2.626333 | K81L | −2.33819 |
| G44W | −3.15754 | N60E | −2.55535 | Q39E | −2.32855 |
| H40Y | −3.05594 | G27S | 2.454676 | R73W | −2.32506 |
| S28Y | −2.88278 | S62P | 2.450937 | T73V | 2.320096 |
| Y58C | −2.83797 | H53L | 2.435279 | T17M | −2.30636 |
| T57K | −2.80295 | Y53P | −2.42282 | W36L | −2.29227 |
| Y58E | −2.75978 | V24G | 2.375488 | P41T | 2.282717 |
| Y32A | −2.73716 | G26R | −2.35902 | S65G | 2.280096 |
| S62F | −2.68586 | W36S | −2.35806 | | |
| P40A | 2.654271 | L20R | 2.357037 | | |

### TSP Classifier

The TSP selected a pair of features: “IGHV4-34@16” and “IGHV44@34.” The overall LOOCV accuracy is 84.1%. The sensitivity is 93.0% and the specificity is 69.2%.

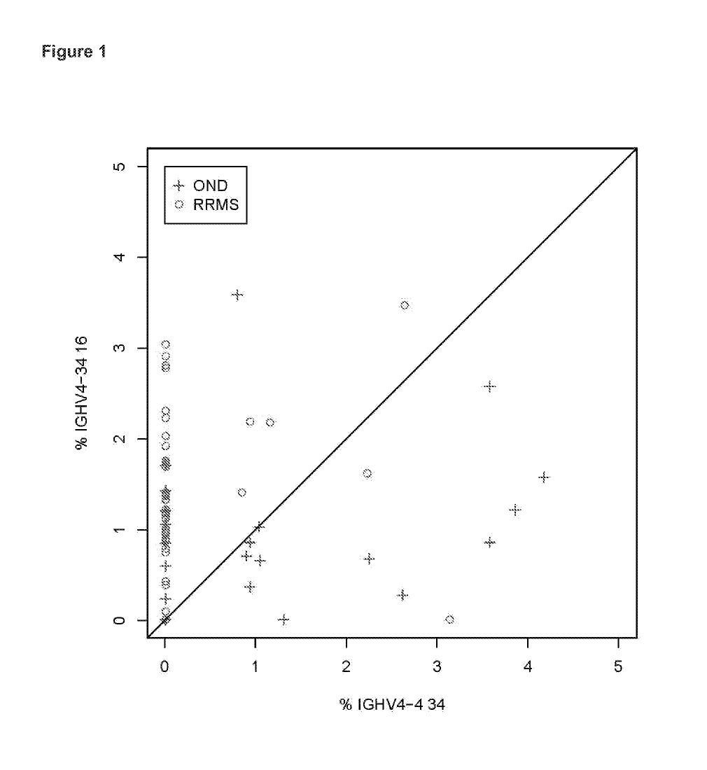

The classification rule has a simple interpretation and is intuitive; when the observed fraction of mutations at “IGHV4-34@16” is greater than that at “IGHV4-4@34” (above the diagonal line), the patient is likely to be RMMS, and vice versa (See FIG. 1).

### Misclassified Patients

TABLE 3
Misclassified patients by three classifiers. Agreement of diagnosis
was reached by an independent panel of three neurologists (Dx by
3), two neurologists (Dx by 2), or by a single neurologist (Dx).

| Batch | Sample | Dx | Reference Dx by 2 | Reference Dx by 3 | k6_v28 | k8_v28 | TSP |
|---|---|---|---|---|---|---|---|
| 1 | 6 | RRMS | Possible RRMS | | x | x | |
| 1 | 7 | OND | Not RRMS, Headache | Not RRMS, Headache | x | x | x |
| 1 | 8 | RRMS | Possible RRMS, CIS | | | | x |
| | | | high risk, TM | | | | |
| 1 | 13 | OND | Not RRMS, Bell's | Not RRMS, Bell's | x | x | |
| | | | palsy | palsy | | | |
| 1 | 15 | OND | Not RRMS, NMO | Not RRMS, NMO | | | x |
| 2 | 3 | RRMS | RRMS | RRMS | | | x |
| 2 | 4 | OND | Not RRMS | | | x | x |
| 2 | 8 | RRMS | RRMS | | | | x |
| 2 | 9 | RRMS | RRMS | RRMS | x | x | |
| 2 | 10 | OND | Not RRMS, Headache | Not RRMS, Headache | | x | |
| 2 | 12 | RRMS | RRMS | RRMS | x | | |
| 2 | 18 | RRMS | Possible RRMS | | x | x | |
| 2 | 23 | RRMS | RRMS | RRMS | | | x |
| 3 | 6 | OND | Not RRMS, Headache | Not RRMS, Headache | | | x |
| 3 | 13 | OND | Not RRMS | | x | x | x |
| 3 | 17 | OND | Not RRMS, NMO | Not RRMS, NMO | x | x | |
| 4 | 2 | OND | Not RRMS, probable | | | | x |
| | | | PPMS | | | | |
| 4 | 7 | OND | Not RRMS | | x | x | |
| 4 | 8 | RRMS | RRMS | RRMS | x | x | |
| 4 | 9 | RRMS | RRMS | | x | | |
| 4 | 11 | OND | Not RRMS, Lyme | Not RRMS, Lyme | | | x |
| | | | Disease | Disease | | | |
| 4 | 20 | OND | Not RRMS | Not RRMS | | | x |

Using a meta-classifier by using majority voting, here will be 10 predictions errors out of 70 training samples. The overall accuracy is then 86%.

The present examples achieve an LOOCV accuracy of up to 86% for predicting RRMS. Several lines of evidence provide additional confidence about the existence of true signals in the data. First, two distinct sets of features (pSUB and pVGENE, both derived from the sequence data) both gave comparable prediction accuracies. Second, the features selected represent both enrichment and depletion of substitutions in the RRMS blood samples (Table 2), as compared to only depletion of substitutions observed previously in the RRMS CSF samples. Third, gene subfamilies are much more evenly represented in these blood samples than in CSF samples. Indeed, greater than 90% of patients have at least ten sequences observed for each of the seven subfamilies (IGHV4-31, IGHV4-30, IGHV4-61, IGHV4-34, IGHV4-39, IGHV4-4, and IGHV4-59). Thus, missing data is no longer a significant issue.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims. Those skilled in the art will recognize, or be able to ascertain using no more than routine experimentation, many equivalents to the specific embodiments of the invention described herein. Such equivalents are intended to be encompassed by the following claims.

All references, to the extent that they provide exemplary, procedural, or other details supplementary to those set forth herein, are specifically incorporated by reference.

1. A method for detecting neurological disease, including immune-mediated neurological disease and/or demyelinating disease, in a patient, the method comprising:
determining in VH4 gene sequences from a patient sample a mutation profile at a plurality of VH4 codon positions, wherein the mutation profile comprises the frequency or percentage of occurrence of a plurality of the following mutations:
S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, S65G; and
classifying the sample fix the presence or absence of neurological disease using a computer-implemented classifier algorithm.

2. The method of claim 1, wherein the plurality of VH4 codon positions is one or more or all of codons 17, 20, 24, 26, 27, 28, 31A, 32, 35, 36, 39, 40, 41, 44, 53, 57, 58, 60, 62, 65, 73, and 81.

3. The method of claim 1 or 2, wherein the neurological disease is multiple sclerosis (MS).

4. The method of claim 1 or 2, wherein the patient has clinically isolated syndrome, and the classifying step confirms MS or rules out MS.

5. The method of any one of claims 1 to 4, wherein the patient is undergoing treatment for MS, and the classifying step determines active disease, disease progression, or disease relapse; or determines disease remission or disease control.

6. The method of any one of claims 1 to 5, wherein the sample is a blood sample, a cerebrospinal fluid sample (CSF), a urine sample; or a fraction thereof.

7. The method of claim 6, wherein the sample is a blood sample.

8. The method of any one of claims 1 to 7, wherein the mutation profile is determined by nucleic acid sequence of VH4 genes in the sample.

9. The method of any one of claims 1 to 7, wherein the mutation profile is determined by quantitative PCR or endonuclease assay.

10. The method of any one of claims 1 to 9, wherein the mutation profile comprises the frequency or percentage of occurrence of at least 5 of said mutations, at least 10 of said mutations, at least 15 of said mutations, at least 20 of said mutations, at least 25 of said mutations, or all of said mutations.

11. The method of any one of claims 1 to 10, wherein the computer-implemented classifier algorithm is a K-Nearest Neighbors algorithm.

12. The method of claim 11, wherein K is at least 4, at least 5, at least 6, at least 7, or at least 8.

13. The method of any one of claims 1 to 12, wherein the classifier algorithm is trained with the frequency or percentage of said mutations in MS patients versus healthy controls and/or patients with other neurological diseases.

14. A method for detecting multiple sclerosis disease in a patient, the method comprising:
determining sequences at codons 17 to 81 in VH4 genes from a patient blood or CSF sample, calculating the frequency or percentage of occurrence of at least five mutations selected from:
S31AL, G44W, H40Y, S28Y, Y58C, T57K, Y58E, Y32A, S62F, P40A, G35A, N60E, G27S, S62P, H53L, Y53P, V24G, G26R, W36S, L20R, K81L, Q39E, R73W, T73V, T17M, W36L, P41T, S65G; and
classifying the sample for the presence or absence of multiple sclerosis by a K-Nearest Neighbors algorithm.

15. The method of claim 14, wherein the patient has clinically isolated syndrome, and the classifying step confirms MS or rules out MS.

16. The method of claim 14, wherein the patient is undergoing treatment for MS, and the classifying step determines active disease, disease progression, or disease relapse; or determines disease remission.

17. The method of any one of claims 14 to 16, wherein the patient sample is a blood sample.

18. The method of any one of claims 14 to 17, wherein the sequences at codons 17 to 81 in VH4 genes are determined by nucleic acid sequencing,

19. The method of any one of claims 14 to 18, wherein the frequency or percentage of occurrence of at least 10 of said mutations, at least 15 of said mutations, at least 20 of said mutations, at least 25 of said mutations, or all of said mutations are calculated.

20. The method of any one of claims 14 to 19, wherein K is at least 4, at least 5, at least 6, at least 7, or at least 8.

21. The method of any one of claims 14 to 20, wherein the K-Nearest Neighbors algorithm is trained with the frequency or percentage of said mutations in MS patients versus healthy controls and/or patients with other neurological diseases.

22. A method for detecting demyelinating disease in a patient, the method comprising detecting in VH4 sequences from a patient sample, mutations at IGVH4-34 codon position 16, and IGHF4-4 at codon position 34, and classifying the patient has having demyelinating disease if the frequency or percentage of mutation at IGVH4-34 codon position 16 is greater than IGH4-4 codon position 34.

23. The method of claim 22, wherein the demyelinating disease is multiple sclerosis (MS).

24. The method of claim 22 or 23, wherein the patient has clinically isolated syndrome, and the classifying step confirms or rules out MS.

25. The method of claim 22 or 23, wherein the patient is undergoing treatment for MS and the method determines active disease, disease progression, or disease relapse, versus disease remission or disease control.

26. The method of any one of claims 22 to 25, wherein the sample is a blood sample, a cerebrospinal fluid sample (CSF), or a urine sample; or fractions thereof.

27. The method of claim 26, wherein the sample is a blood sample.

28. The method of any one of claims 22 to 27, wherein the mutations are detected by nucleic acid sequencing of VH4 genes in the sample.

29. The method of any one of claims 22 to 27, wherein the mutations are detected by hybridization assay, quantitative PCR, or endonuclease assay.

30. The method of any one of claims 1 to 21, wherein a majority voting prediction is made together with the method of any one of claims 22 to 29.
